(19) United States
(12) Patent Application Publication
(10) Pub. No.: US 2024/0393339 A1
(43) Pub. Date: Nov. 28, 2024

(54) METHODS TO PREDICT THE EFFICACY OF NEOADJUVANT ANTI-PD-1 THERAPY IN RESECTABLE ORAL-CAVITY SQUAMOUS CELL CARCINOMA AND TARGET POST-SURGICAL RELAPSES

(71) Applicant: THE REGENTS OF THE UNIVERSITY OF CALIFORNIA, OAKLAND, CA (US)

(72) Inventor: Roger S. Lo, LOS ANGELES, CA (US)

(73) Assignee: THE REGENTS OF THE UNIVERSITY OF CALIFORNIA, OAKLAND, CA (US)

(21) Appl. No.: 18/694,670

(22) PCT Filed: Sep. 23, 2022
(86) PCT No.: PCT/US2022/076963

§ 371 Date: Mar. 22, 2024

Related U.S. Application Data
(60) Provisional application No. 63/261,595, filed on Sep. 24, 2021.

(51) Int. Cl. G01N 33/574; A61K 45/06; A61P 35/00; C07K 16/28

(52) U.S. Cl. CPC G01N 33/57484; A61K 45/06; A61P 35/00; C07K 16/2818

(57) ABSTRACT

Methods of treating resectable head and neck cancer based on analyses of blood and tumor samples collected over the course of a clinical trial and during follow-up after the clinical trial. Omic or multi-plex molecular tools were used to analyze the tissues and identify objective molecular markers and pathogenic mechanisms associated with favorable or unfavorable outcomes. These analyses form the basis for a treatment strategy that improves outcomes by tailoring the treatment to the molecular features of individual patients' blood samples and tumors. This method for personalized treatment distinguishes between subjects who respond to neoadjuvant anti-PD-1/L1 therapy and patients who do not respond to such neoadjuvant therapy, so that treatment is tailored to the subject's responder profile. This approach avoids exposing patients to unnecessary toxicity, and avoids delaying surgical resection when no advantage will be gained by delaying surgery to allow for neoadjuvant therapy.

This application claims benefit of U.S. provisional patent application No. 63/261,595, filed Sep. 24, 2021, the entire contents of which are incorporated by reference into this application.

### ACKNOWLEDGEMENT OF GOVERNMENT SUPPORT

This invention was made with government support under Grant No. CA168585, awarded by the National Institutes of Health. The government has certain rights in the invention.

### BACKGROUND

Head and neck squamous cell carcinoma (HNSCC) ranks sixth among malignancies worldwide. Human papillomavirus (HPV)-negative HNSCC accounts for 75% of HNSCCs and portend far worse prognosis. Oral-cavity squamous cell carcinoma (OCSCC) is a subset of mostly HPV-negative HNSCC. If we can intervene more effectively earlier in the natural history when the disease is still amenable to surgery (i.e., resectable), then we have a chance of improving survival chances or the prognosis of this disease.

Since its first clinical testing, anti-PD-1 immune checkpoint blockade has revolutionized the management of patients with advanced malignancies and is poised to re-shape the multidisciplinary treatment of patients with earlier-stage but high-risk malignancies. Deployment of anti-PD-1 therapy may be relatively more effective against earlier-stage (versus advanced metastatic stage) cancers due to a less evolved cancer and less suppressed immune system. Preclinical experiments support anti-PD-1 therapy in the neoadjuvant (before surgery) compared to the adjuvant (after surgery) setting, presumably because the tumor bulk is critical for therapy-induced antitumor T cell persistence and activity. Clinically, in palpable stage III melanoma where neoadjuvant versus adjuvant combined immune checkpoint blockade was compared, T cell expansion was more vigorous in the neoadjuvant setting.

Because anti-PD-1 is a therapy that re-energizes the patients' tumor-killing immune or T cells, it is thought that deploying this therapy may work better before surgery, when the tumor cells and the killer T cells are still there. The T cells that protect the patients from the tumor cells may thereby persist in the circulation or body of the patient after the surgery to remove residual tumor cells left behind by surgery.

There remains a need for methods to improve the efficacy of immune checkpoint therapy and its coordination with other modes of treatment to improve recurrence free survival in patients with HNSCC.

### SUMMARY

Described herein are improved methods of treating HNSCC based on analyses of tissues (blood and tumor) collected over the course of a clinical trial and during follow-up after the clinical trial. Omic or multi-plex molecular tools were used to analyze the tissues deeply to discover objectively molecular markers and pathogenic mechanisms associated with favorable or unfavorable outcomes. These analyses form the basis for a treatment strategy that improves outcomes by tailoring the treatment to the molecular features of individual patients' blood samples and tumors.

Described herein is the detection of biomarkers at three time points: before neoadjuvant anti-PD-1 therapy, after neoadjuvant anti-PD-1 therapy and at the time of surgery, as well as when patients relapse after both neoadjuvant anti-PD-1 therapy and surgery. Biomarkers were detected in the blood and in the tumors. Potential biomarkers include the types of T cells present, both in the blood and inside the tumors, and genetic changes present inside the tumors. Analysis of recurrent tumors provided insights into targets to prevent or treat relapses.

The methods described herein provide methods of treating a subject in need of treatment for resectable head and neck cancer by distinguishing between subjects who respond to neoadjuvant anti-PD-1/L1 therapy and patients who do not respond to such neoadjuvant therapy, and tailoring the treatment to the subjects' molecular profiles in the peripheral blood or tumor sites. This personalized treatment approach avoids exposing patients to unnecessary toxicity, and avoids delaying surgical resection when no advantage will be gained by delaying surgery to allow for neoadjuvant therapy. Such non-responders may be better served by a more aggressive approach to surgery, and optionally, to chemotherapy, with or without radiation therapy after surgery (adjuvant therapies).

In some embodiments, the method comprises:
(a) assaying a biological sample obtained from the subject for at least one of:
   (1) tumor mutational burden (TMB) in tumor cells,
   (2) mutations in FLT4, CDKN2A, YAP1, and/or JAK2 in tumor cells,
   (3) signature enrichment or mutational status in PTEN in tumor cells,
   (4) ratio of regulatory T to TH17 cells in peripheral blood mononuclear cells (PBMCs),
   (5) scoring of tumor-infiltrating cytolytic T cells,
   (6) T cell receptor (TCR) diversity and clonality in tumors or in PBMCs at a first time point and at a second time point.

The method further comprises (b) treating the subject with:
(1) neoadjuvant anti-PD-1/L1 therapy prior to surgical resection when the assaying of (a) detects one or more “responder markers.” Responder markers include:
   (i) greater than 5.8 mutations per megabase (Mb) in tumor cells,
   (ii) mutations in FLT4 in tumor cells,
   (iii) signature enrichment of PTEN or wildtype PTEN,
   (iv) a ratio of regulatory T cells to TH17 cells of ≤4 in PBMCs,
   (v) an increase of TCR diversity or reduction in TCR clonality in PBMCs at the second time point compared to the first time point,
   (vi) an expansion of large (>5% of total), preexisting (i.e., detected at first time point in the tumor) T cell clonotypes in the tumor at the second time point, and/or
   (vii) brisk tumor infiltration by cytolytic T cells; and
(2) surgical resection without neoadjuvant anti-PD-1/L1 therapy when the assaying of (a) detects one or more “non-responder markers.” Non-responder markers include:
   (i) less than 5.8 mutations per megabase (Mb) in tumor cells,
   (ii) mutations in CDKN2A, YAP1, and/or JAK2 in tumor cells,
   (iii) a ratio of regulatory T cells to TH17 cells of greater than 4 in PBMCs,
   (iv) a reduction in TCR diversity or increase in TCR clonality in PBMCs at the second time point compared to the first time point, and/or
   (v) non-brisk tumor infiltration of cytolytic T cells.

In some embodiments, the assaying of step (a) comprises detecting the ratio of regulatory T to TH17 cells in PBMCs, and is performed prior to anti-PD-1/L1 treatment and prior to surgical resection.

In some embodiments, the method further comprises:
(c) repeating the assaying of (a) at or near the time of surgical resection and after neoadjuvant anti-PD-1/L1 therapy; and
(d) treating the subject with adjuvant anti-PD-1/L1 therapy following surgical resection when the repeated assaying of (c) detects:
   (1) loss of FLT4 mutations,
   (2) loss of PTEN signature enrichment or wildtype status,
   (3) loss of brisk tumor infiltration of cytolytic T cells,
   (4) no significant change or decrease in the diversity of TCR repertoire in PBMCs, and/or
   (5) no gain or expansion in the clonality of preexisting, intratumoral large-size T cell clonotypes.

In some embodiments, the method further comprises co-treating the subject with a combination of anti-PD-1/L1 therapy and agents targeting or neutralizing regulatory T cells, stimulating TH17 cells, or reversing the functional impacts of CDKN2A and JAK2 loss-of-function mutations or YAP1 gain-of-function mutations or post-transcriptional alterations, when the assaying of (a) detects:
(a) mutations in CDKN2A, YAP1, and/or JAK2 in tumor cells,
(b) a ratio of regulatory T cells to TH17 cells of greater than 4 in PBMCs, and/or
(c) non-brisk tumor infiltration of cytolytic T cells.

In some embodiments, the agent that targets regulatory T cells is a CD25NIB antibody. In some embodiments, agents that reverse the functional impacts of CDKN2A and JAK2 loss-of-function mutations or YAP1 gain-of-function mutations or post-transcriptional alterations include agents known to inhibit the YAP1 pathway or to activate innate immunity.

In some embodiments, the co-treating above occurs when the assaying of (a) detects less than 5.8 mutations per Mb in tumor cells.

In some embodiments, the biological sample comprises tumor biopsies and PBMCs isolated from peripheral blood.

In some embodiments, the assaying of (a) is performed before neoadjuvant therapy or at the time of surgery.

In some embodiments, the treating of step (b) and/or (d) further comprises radiochemotherapy.

In some embodiments, the head and neck cancer is HPV-negative squamous cell carcinoma. In some embodiments, the head and neck cancer is oral-cavity squamous cell carcinoma.

### BRIEF DESCRIPTION OF THE DRAWINGS

FIGS. 1A-1F illustrate genomic correlates of innate tumor sensitivity versus resistance and survival in pretreatment tumors. (FIG. 1A) TMBs in responders (n=7) versus non-responders (n=5); p value, Wilcoxon rank-sum test. Hashed dots, median values. (FIGS. 1B and 1C) Kaplan-Meier curves of RFS (1B) and OS (1C) comparing tumors with high TMB (≥median TMB, n=6) versus tumors with a low TMB (<median TMB, n=5); two-sided log rank test. The tumor from individual 12, who was lost to follow-up, was excluded. (FIG. 1D) Genes with recurrent somatic mutations (responders, n=7; non-responders, n=5). Recurrence was defined as non-synonymous mutations in 2 or more individuals and CN alterations in 7 or more individuals. Indel, insertion or deletion; amp, amplification; del, deletion. The status of subject-matched recurrent tumors is shown but not counted toward recurrence. (FIG. 1E) Ratios of variant versus normal allele frequencies in CDKN2A detected in one responder and three non-responders. The CN of CDKN2A is labeled on top. (FIG. 1F) Infiltration levels of CD8⁺ T, T_{REG}, and resting NK cells in FLT4^{WT} (n=508) versus FLT4^{Mut} (n=14) clinical HNSCC tumors from a public dataset in cBioPortal; p values, Wilcoxon rank-sum test. *p<0.05, ***p<0.001.

FIGS. 2A-2E illustrate the evolution of post-operative recurrent tumors. (FIG. 2A) Phylogenetic relationships of subject-specific normal tissue, pretreatment, and recurrent tumors in two responders (individuals 1 and 6) and one non-responder (individual 7). Phylogenetic distances between germline gDNA, most recent common tumor ancestor, pretreatment tumor, and recurrent tumor(s) reflect the number of SNVs and small indels. Select driver genes and their mutations are shown for each evolutionary trajectory. (FIG. 2B) Expression levels of PTEN and JAK2 in pretreatment and recurrent tumors of individual 1. (FIG. 2C) Representative immunofluorescent images merging (1) DAPI (nuclei), pan-cytokeratin (panCK), and PTEN or JAK2 signals from post-treatment and recurrent tumors (individual 1): (2) DAPI (nuclei), panCK, and YAP1 or MDM2 signals from post-treatment and two recurrent tumors (individual 6); and (3) DAPI (nuclei), panCK, and YAP1 signals from post-treatment and recurrent tumors of individual 7 as well as post-treatment tumors (controls) of individuals 9 and 10. Scale bars represent 50 microns, except for MDM2 images (20 μm). (FIG. 2D) Quantification of mIF across whole tissue sections comparing post-treatment versus recurrent tumors in individuals 1, 6, and 7. (FIG. 2E) Images representative of mIF quantifications in (2D). Scale bar, 50 μm.

FIGS. 3A-3C illustrate transcriptomic features of response in pre- and post-treatment tumors. (FIG. 3A) Heatmap showing the top gene sets differentially enriched in responsive versus non-responsive pretreatment tumors (n=11; one pretreatment tumor was excluded because of RNA degradation of its matched post-treatment tumor). (FIG. 3B) Pearson correlation of enrichment scores between PTEN_DN and PPARG signatures in pretreatment tumors (n=11). (FIG. 3C) Heatmap showing top gene sets differentially enriched in responsive versus non-responsive post-treatment tumors (n=11).

FIGS. 4A-4D show post-treatment elevation in systemic TCR diversity and tumoral TCR clonality reflects responsiveness. (FIG. 4A) Gini indices of TCRβ clones in tumors (left) and PBMCs (right) before or after neoadjuvant nivolumab treatment (hashed dots, average values; n=3 per group). Pairwise comparisons by Student's t test, *p<0.05. (FIG. 4B) Pearson correlations of pathologic responses and Gini indices detected in pre- and post-treatment tumors (top) and PBMCs (bottom). (FIG. 4C) Temporal changes in Gini indices within longitudinal tumors (top) or PBMCs (bottom) of each individual (n=3 responders, n=3 non-responders). (FIG. 4D) Pearson correlation of pathologic responses and total clone sizes of preexisting TCR clonotypes in post-treatment tumors.

FIGS. 5A-5E show elevated ratio of T_{REG} to Th17 cells in peripheral blood as a pretreatment marker of non-response. (FIG. 5A) t-distribution stochastic neighbor embedding (t-SNE) map of live cell clusters and immune subpopulations in pre- and post-treatment PBMCs analyzed by CyTOF (n=5 responders, n=4 non-responders, n=4 healthy donors). (FIG. 5B) Heatmap showing the expression values of immune phenotypic protein markers normalized to the maximum mean value across subpopulations. (FIG. 5C) Frequencies of CD4⁺ T cell subpopulations in the total T cell population in responders versus non-responders before or after neoadjuvant nivolumab therapy. p value, Student's t test; **p<0.01. (FIG. 5D) Ratios of frequencies of T_{REG} versus Th17 cells. p value, Student's t test; *p<0.05. (FIG. 5E) Pearson correlations of the pretreatment PBMC T_{REG}/Th17 cell ratios with pretreatment intratumoral levels of CD8⁺ T cells, cytolytic activity signature enrichment, effector T cell signature enrichment, IFNG-6 genes signature enrichment, PD-L1 expression, and Gini indices of TCRβ clonotypes in pretreatment PBMCs or post-treatment tumors.

FIG. 6 provides a schematic illustration of the response patterns to neoadjuvant nivolumab treatment and post-surgical recurrences as explored through analysis of longitudinal tumor and blood samples in a cohort of 12 individuals displaying 33% responsiveness. Pretreatment tumor-based detection of FLT4 mutations and PTEN signature enrichment favors response, and high tumor mutational burden improves recurrence-free survival. In contrast, preexisting and/or acquired mutations (in CDKN2A, YAP1, or JAK2) correlate with innate resistance and/or tumor recurrence. Immunologically, tumor response after therapy entails T cell receptor repertoire diversification in peripheral blood and intratumoral expansion of preexisting T cell clones. A high ratio of regulatory T to T helper 17 cells in pretreatment blood predicts low T cell receptor repertoire diversity in pretreatment blood, a low cytolytic T cell signature in pretreatment tumors, and innate resistance. This discovery provides a molecular framework to advance neoadjuvant anti-PD-1 therapy for individuals with resectable head and neck cancer.

### DETAILED DESCRIPTION

The present disclosure provides new methods for personalized treatment of resectable head and neck cancer by distinguishing between subjects who respond to neoadjuvant anti-PD-1/L1 therapy and patients who do not respond to such neoadjuvant therapy, and tailoring the treatment to the subjects responder profile. This personalized treatment approach avoids exposing patients to unnecessary toxicity, and avoids delaying surgical resection when no advantage will be gained by delaying surgery to allow for neoadjuvant therapy.

### Definitions

All scientific and technical terms used in this application have meanings commonly used in the art unless otherwise specified. As used in this application, the following words or phrases have the meanings specified.

As used herein, “anti-PD-1 therapy” means treatment with an anti-PD-1 antibody (nivolumab/BMS-936558/MDX-1106, pembrolizumab/MK-3475, Pidilizumab), and/or an anti-PD-L1 antibody (BMS-986559, MPDL3280A, and MEDI4736).

As used herein, “neoadjuvant” therapy refers to treatment administered as a first step to shrink a tumor before the main treatment, which is usually surgery, is given.

As used herein, “therapy”, “treatment” or “treating” means any administration of a therapeutic agent according to the present disclosure to a subject (e.g. human) having or susceptible to a condition or disease, such as cancer, for the purpose of: preventing or protecting against the disease or condition, that is, causing the clinical symptoms not to develop; inhibiting the disease or condition, that is, arresting or suppressing the development of clinical symptoms; or relieving the disease or condition that is causing the regression of clinical symptoms. In some embodiments, the term “therapy”, “treatment” or “treating” refers to relieving the disease or condition, i.e. which is causing the regression of clinical symptoms.

As used herein, the term “preventing” refers to the prophylactic treatment of a patient in need thereof. The prophylactic treatment can be accomplished by providing an appropriate dose of a therapeutic agent to a subject at risk of suffering from an ailment, thereby substantially averting onset of the ailment. The presence of a genetic mutation or the predisposition to having a mutation may not be alterable. However, prophylactic treatment (prevention) as used herein has the potential to avoid/ameliorate the symptoms or clinical consequences of having the disease engendered by such genetic mutation or predisposition. It will be understood by those skilled in the art that in human medicine, it is not always possible to distinguish between “preventing” and “suppressing” since the ultimate inductive event or events may be unknown, latent, or the patient is not ascertained until well after the occurrence of the event or events. Therefore, as used herein the term “prophylaxis” is intended as an element of “treatment” to encompass both “preventing” and “suppressing” as defined herein. The term “protection,” as used herein, is meant to include “prophylaxis.” The term “effective amount” refers to that amount of a therapeutic agent that is sufficient to effect treatment when administered to a subject in need of such treatment. The effective amount will vary depending upon the specific activity of the therapeutic agent being used, the severity of the patient's disease state, and the age, physical condition, existence of other disease states, and nutritional status of the patient. Additionally, other medication the patient may be receiving will affect the determination of the effective amount of the therapeutic agent to administer.

As used herein, “pharmaceutically acceptable carrier” or “excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline.

Compositions comprising such carriers are formulated by well-known conventional methods (see, for example, Remington's Pharmaceutical Sciences, 18th edition, A. Gennaro, ed., Mack Publishing Co., Easton, PA, 1990).

As used herein, the term “subject” includes any human or non-human animal. The term “non-human animal” includes all vertebrates, e.g., mammals and non-mammals, such as non-human primates, horses, sheep, dogs, cows, pigs, chickens, and other veterinary subjects. In a typical embodiment, the subject is a human.

As used herein, “a” or “an” means at least one, unless clearly indicated otherwise.

As used herein, a “control” or “reference” sample means a sample that is representative of normal measures of the respective marker, such as would be obtained from normal, healthy control subjects, or a baseline amount of marker to be used for comparison. Typically, a baseline will be a measurement taken from the same subject or patient. The sample can be an actual sample used for testing, or a reference level or range, based on known normal measurements of the corresponding marker.

### Assays

Assaying tumor mutational burden (TMB) in tumor cells is determined by sequencing tumor cells obtained from a biological sample, such as a biopsy. The TMB is defined as the total number of nonsynonymous mutations per coding area of a tumor genome. While TMB can be determined using whole exome sequencing, targeted panel sequencing is a more cost- and time-efficient method to measure TMB. In some embodiments, a TMB of 5.8 mutations per megabase (Mb) in tumor cells is a cutoff point. For example, a TMB greater than 5.8 favors neoadjuvant anti-PD-1/L1 therapy prior to surgical resection, while a TMB below 5.8 favors surgical resection without neoadjuvant anti-PD-1 therapy.

Assays for detecting mutations in FLT4, CDKN2A, YAP1, and/or JAK2 in tumor cells, and signature enrichment or mutational status in PTEN in tumor cells, can be performed using conventional genomic (gDNA sequencing, whole exome sequencing) and transcriptomic (RNA sequencing) methods.

Assays for detecting the ratio of regulatory T to TH17 cells in peripheral blood mononuclear cells (PBMCs) can include, for example, cytometry by time of flight, or CyTOF. CyTOF uses mass cytometry to quantify labeled targets of single cells, as described in Example 1 herein.

Assays for scoring of cytolytic T cells infiltrating tumors can be performed using RNA sequencing data, as described in Example 1 herein. For analyzing T cell infiltration into tumor biopsies, a suitable assay is Immunoscore, which categorizes such infiltration as high or low based on specific histologic criteria. Immunoscore measures the density of two T lymphocyte populations (CD3/CD8, CD3/CD45RO or CD8/CD45RO) in the center and at the periphery of the tumor. The Immunoscore provides a score ranging from 0 (10) when low densities of both cell types are found in both regions, to Immunoscore 4 (14) when high densities are found in both regions. (See, e.g., Marliot F, et al. Immunoscore assay for the immune classification of solid tumors: Technical aspects, improvements and clinical perspectives. Methods Enzymol. 2020; 636:109-128. doi: 10.1016/bs.mie.2019.07.018. Epub 2019 Oct. 18. PMID: 32178816.)

Other strategies for scoring tumor infiltrating lymphocytes (TIL) that have been employed for melanoma tissue include the system devised by Clark et al., and the approach of the Melanoma Institute Australia (MIA). The Clark scoring system defines three distinct TIL patterns as absent, non-brisk and brisk. “Absent” indicates when no TIL are present or they do not infiltrate the tumor. “Non-brisk” denotes one or more scattered foci of lymphocytes. “Brisk” describes a diffuse infiltration of lymphocytes throughout the tumorigenic vertical growth phase or along the base of the tumor. Clemente et al. further divided the scoring of brisk TIL patterns into peripheral (along the tumor base) or diffuse (infiltrating the entire invasive portion of the tumor). Generally, perivascular lymphocytes and lymphocytes in regions of fibrosis are not included in the scoring. The Clark system remains in wide usage due to its reproducibility, ease of application and strong interobserver agreement.

Assays for detecting T cell receptor (TCR) diversity and clonality in tumors or in PBMCs can be performed using, for example, an Immunoseq assay (e.g., ImmunoSeq hsTCRβ kit; Adaptive Biotechnologies, Seattle, WA). TCR diversity and/or clonality can be compared to a reference level, or assayed at a first time point and at a second time point, and optionally, at subsequent time points, for the same subject. Clonotypes can be defined by unique CDR3 amino acid sequences. The clonality of TCR repertoires can be estimated through calculating the Gini-Simpson index by R package tcR. For an individual subject, measuring statistically significant changes from two time points (e.g., before neoadjuvant treatment and then at the time of surgery, in the PBMCs and tumor site) can be used to detect increasing or decreasing TCR diversity and/or clonality.

### Kits & Compositions

Provided are kits and/or compositions comprising one or more reagents and/or therapeutic agents suitable for use in the methods described herein, and optionally, one or more suitable containers containing reagents and/or agents of the invention. Such kits can comprise a carrier, package or container that is compartmentalized to receive one or more containers such as vials, tubes, and the like, each of the container(s) comprising one of the separate elements to be used in the method. The reagents and/or agents of the kit may be provided in any suitable form, including frozen, lyophilized, or in a pharmaceutically acceptable buffer such as TBS or PBS. The reagents can include, for example, reagents that detect one or more of the responder markers and/or non-responder markers described herein.

Agents include an anti-PD-1 antibody, and/or an anti-PD-L1 antibody. Examples of anti-PD-1 antibodies include, but are not limited to, nivolumab/BMS-936558/MDX-1106, pembrolizumab/MK-3475, and Pidilizumab. Examples of anti-PD-L1 antibodies include, but are not limited to, BMS-986559, MPDL3280A, and MEDI4736. Agents can be provided in the form of a composition suitable for administration to a subject in accordance with the methods described here.

Treatment with compositions can be administered in a single dose or as a series of doses administered over time. Dosage and treatment regimens can be determined by the treating physician, taking into account disease severity, patient condition, and other factors.

The kit of the invention will typically comprise the container(s) described above and one or more other containers comprising materials desirable from a commercial and user standpoint, including buffers, diluents, filters, needles, syringes, and package inserts with instructions for use. In addition, a label can be provided on the container to indicate that the composition is used for a specific application, and can also indicate directions for use, such as those described herein. Directions and or other information can also be included on an insert, which is included with the kit.

### EXAMPLES

The following examples are presented to illustrate the present invention and to assist one of ordinary skill in making and using the same. The examples are not intended in any way to otherwise limit the scope of the invention.

### Example 1: Response and Recurrence Correlates in Patients Treated with Neoadjuvant Anti-PD-1 Therapy for Resectable Oral-Cavity Squamous Cell Carcinoma

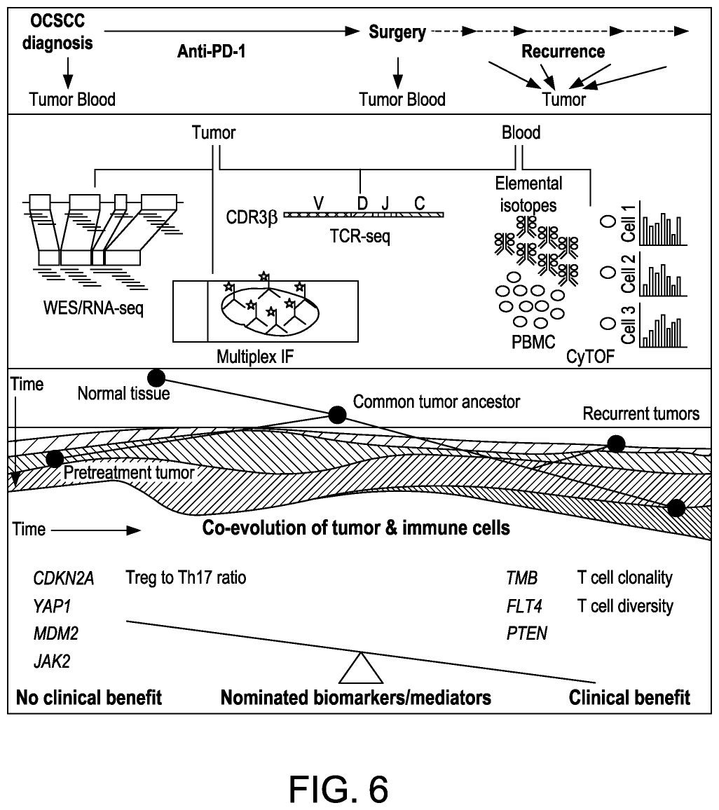

This Example is summarized in FIG. 6. Neoadjuvant PD-1 blockade is efficacious in some patients with high-risk, resectable oral-cavity, head-and-neck cancer. To explore correlates of response patterns to neoadjuvant nivolumab treatment and post-surgical recurrences, we analyzed longitudinal tumor and blood samples in a cohort of 12 patients displaying 33% responsiveness. Pretreatment tumor-based detection of FLT4 mutations and PTEN signature enrichment favors response, and high tumor mutational burden improves recurrence-free survival. In contrast, preexisting and/or acquired mutations (in CDKN2A, YAP1, JAK2) correlate with innate resistance and/or tumor recurrence. Immunologically, tumor response after therapy entails T-cell receptor repertoire diversification in peripheral blood and intratumoral expansion of preexisting T-cell clones. A high ratio of regulatory to T helper 17 cells in pretreatment blood predicts innate resistance, low cytolytic T-cell signature in pretreatment tumor, and low T-cell receptor repertoire diversity in pretreatment blood. This provides a molecular framework to advance neoadjuvant anti-PD-1 therapy for patients with resectable head-and-neck cancer.

Since its first clinical testing¹, anti-PD-1 immune checkpoint blockade has revolutionized the management of patients with advanced malignancies and is poised to re-shape the multidisciplinary treatment of patients with earlier-stage but high-risk malignancies. Deployment of anti-PD-1 therapy may be relatively more effective against earlier-stage (versus advanced metastatic stage) cancers due to a less evolved cancer and less suppressed immune system. Preclinical experiments support anti-PD-1 therapy in the neoadjuvant (before surgery) compared to the adjuvant (after surgery) setting², presumably because the tumor bulk is critical for therapy-induced antitumor T cell persistence and activity. Clinically, in palpable stage III melanoma where neoadjuvant versus adjuvant combined immune checkpoint blockade was compared³, T cell expansion was more vigorous in the neoadjuvant setting.

Head and neck squamous cell carcinoma (HNSCC) ranks sixth among common epithelial malignancies worldwide.⁴ Human papillomavirus (HPV)-positive and -negative categories of HNSCC have distinct multi-omic, clinical, and therapeutic response characteristics, with HPV-negative HNSCC accounting for 75% of all HNSCCs and portending far worse prognosis. Historically, over a third of patients, in particular those with HPV-negative HNSCC, relapse despite intensive postoperative (adjuvant) chemoradiotherapy.⁶ Compared to HPV-positive HNSCC, HPV-negative HNSCCs harbor more mutations and display heightened chromosome instability.⁷ Oral-cavity squamous cell carcinoma (OCSCC) as a subsite consists of mostly HPV-negative HNSCC.

Anti-PD-1 therapy with nivolumab or pembrolizumab improves the overall survival of patients with platinum-resistant recurrent and metastatic HNSCC, including OCSCC.⁸⁻¹⁰ With response rates around 20% and a survival benefit compared with chemotherapy, additional therapeutic strategies are needed. The potential clinical benefit of neoadjuvant anti-PD-1 therapy has been explored in small cohorts with resectable, locally advanced, HPV-negative HNSCC.^{11,12} In one correlative study using PD-L1 immunofluorescence, pretreatment PD-L1 was not correlated with volumetric or pathologic response among 12 patients who received two doses of neoadjuvant nivolumab.¹¹ In another correlative study using genomic techniques (whole-exome- and RNA-seq), pretreatment tumor mutational burden (TMB) did not correlate with the extent of pathologic response among 24 patients who received one dose of neoadjuvant pembrolizumab.¹²

Here, using longitudinal blood and tumor tissues obtained from 12 patients based on a clinical trial we carried out¹³, we set out to generate hypotheses regarding tumor cell-intrinsic and immunologic mechanisms of response patterns, survival, and post-operative recurrence. By analyzing data from each platform and integrating multi-omic data, we dissect temporal relationships between mutational and transcriptomic alterations as well as systemic and intratumoral immunity. Thus, this Example provides insights into tumor and immune cell co-evolution in OCSCCs treated with neoadjuvant anti-PD-1 therapy and identify potential predictive biomarkers and/or mechanisms of response, resistance, and post-surgical recurrence.

Throughout this Example, where reference is made to Supplemental Materials, e.g., Tables S1-S4, Figures S1-S5, these materials, along with a table listing all resources used herein, are available at doi.org/10.1016/j.xcrm.2021.100411.

### Methods

### Specimen Collection, Clinical Data, and Multi-Omic Data

We collected tissue (peripheral blood, tumor) samples from 12 patients with OCSCC who were treated with neoadjuvant nivolumab therapy after local IRB approval. Informed consent was obtained from all patients. The clinical characteristics as well as radiographic and pathological measurements of tumor sizes are in Table S1 and S2. The designs of associated clinical trial and this correlative study are shown in Figure S1. Table S3 displays the list of tissues collected and multi-omics analyses performed in this study.

### Whole Exome Sequencing (WES) and RNA Sequencing (RNA-Seq) Data Generation

Genomic DNA (gDNA) and total RNA were extracted from snap frozen tumor tissue using the QIAGEN AllPrep DNA/RNA Mini Kit and the Ambion mirVana miRNA Isolation Kit. Formalin-fixed paraffin-embedded (FFPE) tumor tissues were extracted for FFPE gDNA using the QIAGEN QIAamp DNA FFPE Tissue Kit. Patient-matched normal genomic DNA from viably frozen peripheral blood mononuclear cells (PBMCs) were extracted using the QIAGEN FlexiGene DNA Kit. Frozen tissue-derived and FFPE tissue-derived gDNA libraries were constructed using the Roche Kapa HyperPlus Library Preparation Kit. Briefly, after enzymatic fragmentation of gDNA, the libraries were constructed by end repairing and A-tailing the fragmented DNAs, ligation of adapters, and PCR amplification. After library construction, indexed frozen tissue-derived and FFPE tissue-derived libraries were separately pooled and then hybridized using SeqCap EZ HyperCap Workflow v2.1 and Kapa HyperCap Workflow v3.0, respectively, followed by PCR amplification. Finally, indexed DNA libraries were quantified for equal molar pooling and paired-end sequenced with a read length of 2×150 bp on the Illumina NovaSeq 6000 S4 platform. RNA libraries were constructed using the NuGEN Universal Plus mRNA-Seq with NuQuant Library Preparation Kit to enrich for all poly (A) transcripts within the transcriptome. Briefly, after RNA fragmentation, double-stranded cDNAs were generated using a mixture of random and oligo (dT) priming. Then the libraries were constructed by end repairing the cDNAs to generate blunt ends, ligation of unique dual index (UDI) adapters, followed by strand selection and PCR amplification. Finally, indexed cDNA libraries were quantified for equal molar pooling and paired-end sequenced with a read length of 2×150 bp on the Illumina NovaSeq 6000 S4 platform. In total, 16 tumors from 12 patients and patient-matched normal PBMC samples were subjected to WES, and 23 tumors from 11 patients were subjected to RNA-seq.

### WES and RNA-Seq Data Processing

### Somatic Mutation Calling, Copy-Number Analysis, and Phylogeny

In total, 16 tumors from 12 patients and matched normal blood samples were process to generate WES. Sequencing was performed using paired-end sequencing with a read length of 2×150 bps based on the NovaSeq V4 platform. We conducted somatic variant calling for single-nucleotide variants (SNVs) and small insertion-deletions (INDELs) as we previously reported20,35,49-52. Mutations were then annotated by using Oncotator53. Tumor purity, ploidy, and somatic copy-number alterations (CNAs) were detected by Sequenza⁵⁴. Characteristics of WES data are summarized in Table S4. The phylogenetic analysis was performed using the PHYLIP program with the parsimony algorithm, as previously reported⁵¹.

### HLA Genotyping and Mutation Calling

HLA typing for each patient was inferred based on normal blood WES data using the POLYSOLVER algorithm16. HLA mutation calling for HLA-A, HLA-B, and HLA-C genes was performed by using the POLYSOLVER-based mutation detection pipeline from the Broad Institute's Polysolver Docker container, which is available at software.broadinstitute.org/cancer/cga/polysolver_run.

### RNA-Seq Data Analysis

We analyzed the paired-end 2×150 bp RNA-seq data according to the pipeline we previously reported recently⁴⁹. Briefly, paired-end transcriptome reads were mapped to the Genome Reference Consortium Human Build 37 (GRCh37) reference genome using HISAT2⁵⁵, and then gene-level counts were estimated by the htseqcount program⁵⁶. The normalized expression level of each gene, TPM, was calculated by the R package GeoTcgaData. By using the R package GSVA⁵⁷, we performed single-sample gene set enrichment analysis (ssGSEA) to generate the absolute enrichment scores of the collections of gene sets from the Broad Institute's Molecular Signatures Database (C2 oncogenic gene sets and C7 immunologic gene sets) and gene signatures previously reported to be associated with ICB response. TPM values were used as input into the GSVA program using the default ‘kcdf=Gaussian’ option. Differentially enriched gene sets between the responder vs. non-responders pre- and post-treatment samples were defined by the sum of differences in enrichment scores being greater than 0.3 and a t-test P value being less than 0.05. CIBERSORTx6⁵⁸ was used in the ‘absolute mode’ to estimate infiltration levels of 22 immune cell types with TPM values as the input.

### Analysis of Public Genomic Data Sets

We downloaded the normalized gene expression level of an RNA-seq dataset of

HNSCC patients (Head and Neck Squamous Cell Carcinoma; TCGA, Firehose Legacy) from cBioPortal. CIBERSORTx was used to estimate the abundance of 22 immune cell types with the normalized expression level as an input. The status of the FLT4 genotype (FLT4Mut or FLT4^{WT}) in each patient was obtained from cBioPortal, and then mapped to patient IDs in the RNA-seq dataset. Group comparison between FLT4Mut vs. FLT4^{WT} patients was performed in the enrichment level of each immune cell type with Wilcoxon rank sum test.

Mass Cytometry (CyTOF) Data Generation from PBMCs

After thawing, PBMCs were live/dead stained with 200 μM Rh-103 (Fluidigm) for 2 minutes at room temperature. To achieve increased throughput and homogenous staining, metal cell barcoding against human immune CD45-positive cells was used. The metal isotopes (Trace Sciences International, Richmond Hill, ON, Canada) used for barcoding were: 105Pd, 106Pd, 108Pd, 111 In, 115 In, 194 Pt, 195 Pt, 196 Pt, and 198 Pt. Metal barcoding reagents were prepared by combining 2 molar equivalents of isothiocyanobenzyl-EDTA (Dojindo Molecular Technologies, Rockville, MD) with 1 molar equivalent of metal chloride in ammonium acetate buffer (20 mM, pH 6.0). Chelated metal solutions were immediately lyophilized and dissolved in DMSO at 10 mM final concentration for long-term storage at −20° C. Pd-loaded SCN-Bn-EDTA stock was thawed and 6.4 μL were added to 100 μg the anti-human CD45 antibody (clone: HI30) dissolved in a total of 313 μL PBS, mixed by pipetting and incubated for 1 h at 37° C. The conjugate was washed at least three times with 300 μL PBS over a 50 kDa spin filter for 10 min at 4° C. and 12,500×g, then transferred to a 1.6 mL microcentrifuge tube. Protein concentration was quantified by Nanodrop (Thermo Fisher, Waltham, MA, USA) at 280 nm, antibody stabilizer (Candor Biosciences, Wangen, Germany) was added to the preparation at a 1:1 ratio, and antibodies were kept at 4° C. Barcoding reagents were titrated to achieve optimal labeling. A unique to each sample combination of exactly 3 metal cell barcoding reagents diluted in 300 μL PBS was added and then incubated for 20 min at room temperature. Cells were washed twice with 1 mL PBS at 4° C. Barcoded cells were then combined in a single tube and washed with cell staining buffer (CSB, PBS+0.5% BSA+2 mM EDTA). Surface proteins were stained with antibodies at 37° C. for 20 minutes and for an additional 10 minutes at 4° C. Cells were washed in CSB and incubated over night with 250 nM iridium intercalator (Fluidigm) in Maxpar cell fix/perm buffer (Fluidigm) to label cellular DNA. Subsequently, cells were washed with PBS followed by distilled water and resuspended in 10% EQ beads (Fluidigm) in distilled water. Mass cytometry acquisition was performed on a CyTOF2.1 (Helios) mass cytometer (Fluidigm).

### CyTOF Data Analysis

Mass cytometry flow cytometry standard (FCS) data files were concatenated, bead-normalized, and debarcoded using Helios software (Fluidigm). Data were then exported into individual files for each sample. Total live cell populations were manually identified and exported using negative and positive gating strategies in Cytobank⁵⁹. We applied Cytofkit⁶⁰ to perform the t-Distribution Stochastic Neighbor Embedding (t-SNE) analysis separately on the manually gated live cell populations. We selected 5,000 in each sample to ensure equal representation of cells across samples. All the cell lineage markers in the immune panel were used in clustering analysis. We chose 3,000 iterations, perplexity of 30, and theta of 0.5 as the standard t-SNE parameters. Mean intensity values of markers in each cluster were calculated and visualized via heatmaps. Cells were assigned to different functional populations on the basis of the local gradient expression of known cell lineage markers. Based on expression of known marker genes, clusters were annotated as MHC II-classical monocytes (CD14+CD11b+CD16−HLA−DR−), MHC II+ classical monocytes (CD14+CD11b+CD16−HLA−DR+), non-classical monocytes (CD14+CD11b+CD16+), dendritic cells or DCs (CD33+CD11c+HLA−DR+), B cells (CD19+), T cell subsets (naïve or TN, CD45RA+CD62L+CCR7+CD45RO−; effector memory or TEM, CD45RA−CCR7−CD45RO+; central memory or TCM, CD45RA−CCR7+CD45RO+; T terminally differentiated or TTD, CD45RA+CCR7−CD27−CD28−; regulatory T or T_{REG}, CD4+FOXP3+; T helper 2 or TH2, CD4+CCR4+; T helper 17 or TH17, CD4+CD26hi; Gamma delta or γδTC, CD3+TCRgd+), NK1 (CD3+CD94+CD16+CD62L−) and NK2 (CD3+CD94+CD16+CD62L+). The percentages of different immune cell subsets were calculated for each sample. We defined a T_{REG}/TH17 ratio as the fold change of frequencies between T_{REG} vs. TH17.

### Generation and Analysis of TCR-Seq Data

Genomic DNA (gDNA) was isolated from patient-matched PBMCs and tumor tissues using Maxwell RSC DNA from Cells and DNA from Tissue kits, respectively (Promega, Madison, WI). TCRβ libraries were prepared using the ImmunoSeq hsTCRβ kit (Adaptive Biotechnologies, Seattle, WA) according to manufacturer's instructions. Briefly, TCRβ libraries were generated from PBMC gDNA samples (480 ng input DNA except for matched samples from Pt7 at 310.4 ng input DNA) for deep sequencing (6 replicates per sample, except for Pt7 post-treatment, for which 5 replicates were generated due to limited gDNA recovery) and tumor gDNA samples (4.8 mg input DNA except for matched samples from Pt4 at 1.44 mg input DNA and matched samples from Pt9 at 1.96 mg input DNA) for survey sequencing (2 replicates per sample). Final libraries were pooled at a concentration of 3 nM and sequenced on an Illumina NovaSeq 6000 S4 flow cell at VANTAGE (Vanderbilt University, Nashville, TN).

We performed pre-processing and quality control of the raw data by using the immunoSEQ analyzer (Adaptive Biotechnologies, Inc.). We then exported measurement metrics of processed data into the tsv file. Only the productive rearrangements and the corresponding productive CDR3 amino acid sequences were considered for downstream analysis. Clonotypes were defined by unique CDR3 amino acid sequences. The clonality of TCR repertoires was estimated through calculating the Gini-Simpson index by R package tcR⁶¹.

### Survival Analysis

Survival analysis for recurrence-free survival (RFS) and overall survival (OS) were compared via the two-sided log-rank test by R package survival. We compared RFS and OS in responders vs. non-responders, and patients with a high level vs. a low level of a certain factor. High or low levels of a certain factor were defined by the value of the factor ≥median value or value of the factor <median value across the cohorts.

### Immunofluorescence (IF) Analysis

Tumor tissues were fixed in formalin followed by paraffin-embedding. After deparaffinization and rehydration, tissue sections were antigen-retrieved by heat. Permeabilization and blocking were followed by overnight incubation with primary antibodies [pan-cytokeratin (Abcam, ab27988), PTEN (Genetex, GTX101025)], JAK2 (Abcam, ab 108596), YAP1 (Abcam, ab52771), and MDM2 (Cell Signaling Technology, 86934). IF was performed with Alexa Fluor-conjugated secondary antibodies (Life Technologies, A-11029, A-21429). Nuclei were counterstained by DAPI. Signals were captured with a Zeiss microscope (AXIO Imager A1) mounted with a charge-coupled device camera (Retiga EXi QImaging), and the images captured by Image-pro plus 6.0. Representative images are shown. Digitized images of whole-slide staining are available upon request.

### Multiplex IF Analysis

Multiplex immunofluorescence (mIF) was performed utilizing Ventana Discovery Ultra (Roche) and Opal fluorophores (Akoya Biosciences). Five micrometer-thick tissue sections on Superfrost microscopic slides (VWR International) were deparaffinized using EZ-Prep reagent (Roche) followed by antigen retrieval in CC1 buffer (pH 9, 95° C.; Roche). Discovery Inhibitor (Roche) was applied to inhibit enzymatic activities followed by 6 sequential rounds of staining. Each round included the addition of a primary antibody followed by detection using the OmniMap secondary antibody (Roche). Signal amplification was performed utilizing Opal fluorophores in the conditions suggested by the manufacturer. Between rounds of staining the tissue sections underwent heat-induced epitope retrieval to remove the primary-secondary-HRP antibody complexes before staining with the subsequent antibody. The primary antibodies and corresponding fluorophores are PanCK (DAKO) in Opal 480; PD-L1 (Cell Signaling) in Opal 520; CD68 (DAKO) in Opal 570; Granzyme B (Leica) in Opal 620; CD8 (Leica) in Opal 690, and CD3 (Roche) in Opal 780. The slides were then counterstained with Spectral DAPI (Akoya Biosciences) and mounted with ProLong Diamond antifade mounting medium (Thermo Fisher Scientific).

Stained slides were imaged using the Vectra Polaris imaging system (Akoya Biosciences). A whole slide scan was acquired with 20× resolution. Following image capture, regions of interest (ROIs) were selected on each slide using the Phenochart viewer (Akoya Biosciences) and imported into the inForm software (Akoya Biosciences) followed by unmixing the spectral libraries, cell segmentation and cell phenotyping. ROIs corresponding to whole tumor regions from each slide were then analyzed to identify and characterize the cells. The data was then exported and graphed with Prism (Graphpad). Representative images were exported using inForm software following spectral unmixing.

### Results

Clinical Characteristics Associated with Tissues

We obtained tissues from patients (n=11) who enrolled in a single-arm, investigator-initiated, single-institution phase II clinical trial (NCT03021993) of OCSCC and from one additional patient (patient #8 or Pt8) (total n=12) who fell out of eligibility due to rapid progression. Detailed eligibility and inclusion/exclusion criteria are described in a related manuscript.¹³ Patient and disease characteristics are summarized in Table S1. Enrollment of patient subjects in the trial and participations in tissue biopsies and collections were approved by the local Institutional Review Board. Briefly, we performed (i) time-of-flight mass cytometry (CyTOF) on pre- and post-treatment peripheral blood mononuclear cells (PBMCs), (ii) whole-exome sequencing (WES) on patient-matched normal, pretreatment tumors, and, when applicable, post-operative recurrent tumors, (iii) RNA-seq on patient-matched pre- and post-treatment tumors and, when applicable, post-operative recurrent tumors, and (iv), genomic DNA (gDNA)-based T cell receptor (TCR)-seq on patient-matched pre- and post-treatment PBMCs and tumors in a subset of responders vs. non-responders.

Design of the trial and tissue collection is schematized in Figure S1. Briefly, primary tumors were required to be from patients with systemic and radiation treatment-naive stage II to IVA OCSCC to ensure response to therapy could be accurately assessed clinically and radiographically. Patients included in this study received 3-4 biweekly doses of 3 mg/kg nivolumab (except one patient who received only 2 biweekly doses) followed by definitive surgical resection with curative intent. Radiographic tumor size was defined as the greatest cross-sectional dimension of the tumor on the enrollment imaging study, and post-treatment size was the greatest cross-sectional dimension of the tumor on surgical pathology. Interval radiographic evaluation occurred after a total of three doses of nivolumab and between days 28-35. Disease progression determined at interval radiographic evaluation (greater than 20% increase in tumor size) were treated with definitive surgical resection between days 36-42. In the event of stable disease or response, patients received a 4^{th} dose of nivolumab on day 43+/−1 followed by definitive surgical resection on day 50-56. Objective response rate was defined as pathologic complete+pathologic partial response (>30% reduction in tumor size of the surgical specimen). Change in size was calculated by comparing the pre-nivolumab radiographic measurement (single greatest dimension) with the final pathologic measurement (Table S2). For this correlative study, we used the change in tumor size, based on the pretreatment radiographic measurement and the pathologic measurement after definitive surgery. Given the short duration of therapy, responders were defined as patients who derived clinical benefit (complete response, partial response, and stable disease per RECIST 1.1), and non-responders were defined as patients who derived no clinical benefit (progression per RECIST 1.1). At the cutoff of this multi-omic study, the median follow-up is 2.05 years. Patient-matched and longitudinal tumor and PBMC tissues analyzed by multi-omics are summarized in Table S3.

### Pretreatment Tumor Genomic Features of Response Patterns and Survival

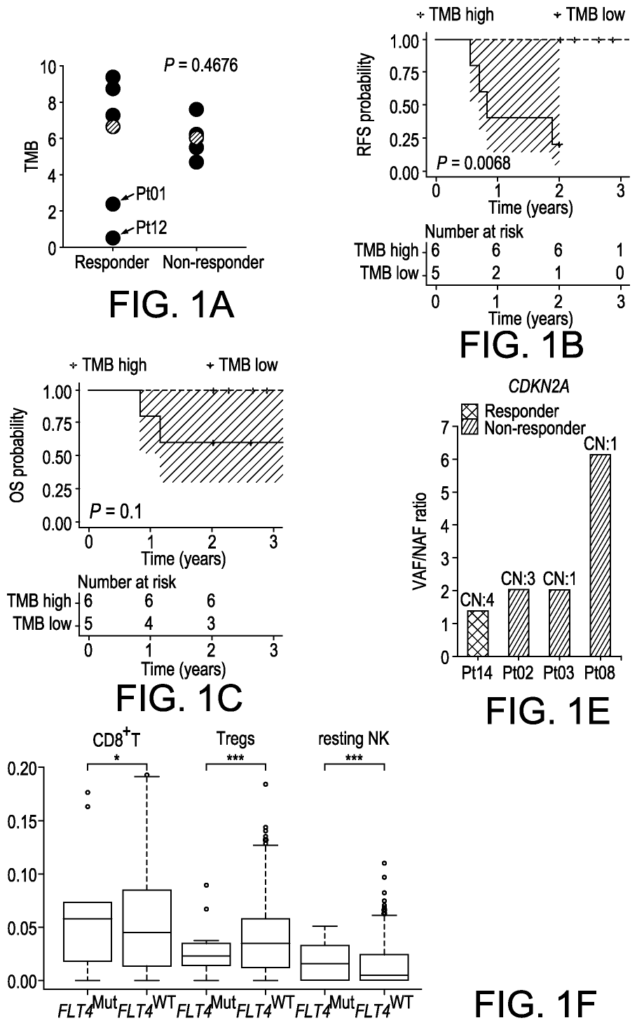

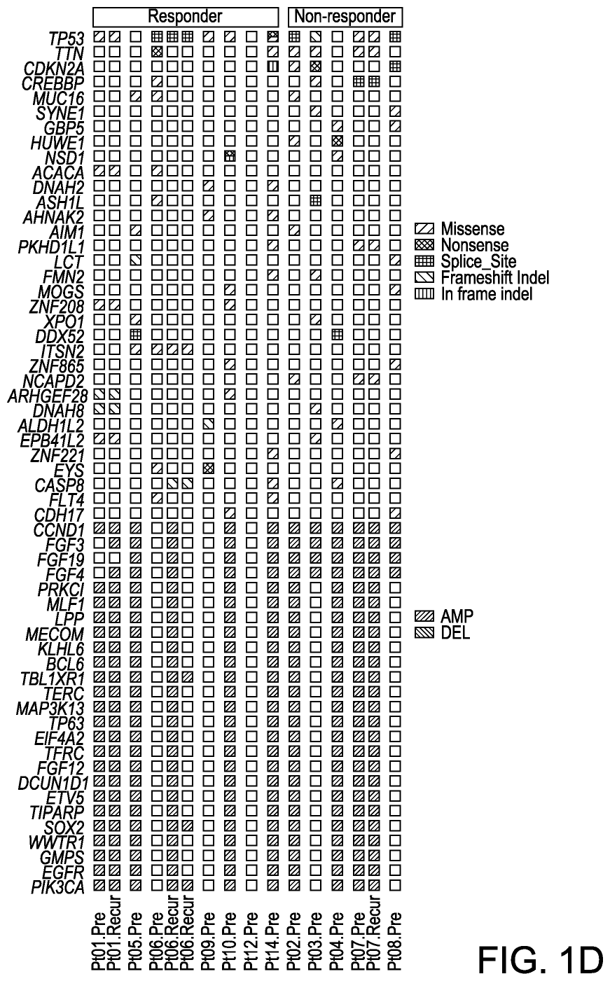

We analyzed WES from 16 tumors (12 pretreatment and 4 recurrence) and matched blood samples from 12 patients. The average number of mutations per tumor was 347 (range, 31-559), corresponding to a mean tumor mutational burden (TMB) of 5.79 mutations/MB (range, 0.52-9.32), which is typical for HNSCC (Table S4). We observed no significant difference in the TMB of pretreatment tumors from responders vs. non-responders (median 6.63 vs. 5.98 SNVs/MB, respectively; P=0.4676, Wilcoxon rank-sum one-side test, FIG. 1A). Among responsive pretreatment tumors, Pt12's tumor harbored a very low TMB (0.52 SNVs/MB), although its response was categorized as stable disease, and the recurrence-free survival (RFS) of Pt12 was <0.3 years (Table S2; OS, not available due to loss of follow-up). In contrast, high TMB (defined as the upper half of the median) in the pretreatment tumor was significantly associated with improved RFS (FIG. 1B) but not with improved OS (FIG. 1C) (median follow-up of 2.05 years). Moreover, tumor response, as defined, did not associate with improved RFS (Figure S2A) or OS (Figure S2B). Neither purity nor ploidy was correlated with a pathologic response (Figure S2C).

HLA-I homozygosity was correlated with poor response and reduced overall survival in advanced melanoma and non-small cell lung carcinoma patients treated with immunotherapies.¹⁴ We observed a non-significant association in the opposite direction, as HLA-I (HLA-A, HLA-B and HLA-C) homozygosity in at least one locus was of a higher proportion in responder group (4 of 7 in responders vs. 0 of 4 in non-responders; Fisher-exact test. P=0.0808; Figure S2D). Additionally, there was no evidence of HLA-I mutations being associated with responsiveness (Figure S2D). HNSCCs have been shown to harbor relatively high levels of somatic changes in HLA class I genes¹⁵, and hotspot mutations in HLA I genes have been associated with upregulation of signatures of effector T cell cytolytic signatures.¹⁶ Our results suggest that MHC-I activity/diversity may not be an important factor driving innate anti-PD-1 resistance in early-stage and locally advanced OCSCCs.

Next, we identified non-synonymous (missense, nonsense, frameshift) mutations, splice site mutations, in-frame insertion/deletions (indel) as well as amplifications and deletions. We then visualized the genes mostly recurrently impacted by these mutations among the pretreatment tumors (FIG. 1D). We observed well-known significantly mutated genes in HNSCC, including TP53 (75%), CDKN2A (33.3%), and CREBBP (25%). We also identified frequently amplified genes, e.g., CCND1 (75%), MAP3K13, PIK3CA, EGFR, and SOX2 (58.3%). Non-synonymous mutations in CDKN2A were detected in three of five non-responding tumors (H83Y in Pt2; R80* in Pt3; splice site mutation in Pt8), in contrast to one of seven responding tumors (in-frame deletion in Pt14) (FIG. 1D). This mutation frequency (60%) in the non-responders is higher compared with the background mutation rate of 20.32% (291 of 1452 HNSCC tumors in cBioProtal, Fisher exact test, P=0.0590; Benjamin-Hochberg adjusted P=0.0861). Also, the ratios of variant to normal allele frequencies of CDKN2A are elevated among the responders, driven in part by deletion of the wild type copy (Pt3 and Pt8) and selective amplification of the mutant copy (Pt2) (FIG. 1E). Interestingly, FLT4 was exclusively mutated in responsive tumors (2 of 7 tumors) (FIG. 1D). Given the background mutation rate of 2.20% (32 of 1452 HNSCC tumors in cBioPortal), FLT4 was mutated more frequently than expected (Fisher-exact test, P=0.0103; Benjamin-Hochberg adjusted P=0.0515). We estimated immune cell proportions from a public RNA-seq dataset of HNSCC in cBioPortal. We identified gene expression specific to three immune cell types to be significantly differentially expressed between FLT4^{MUT} and FLT4^{WT} tumors. Notably, CD8⁺ T cells and resting NK cells were elevated, whereas T_{REG} cells was lower in FLT4^{MUT} tumors (FIG. 1F).

Clonal Evolution of Recurrence after Neoadjuvant Anti-PD-1 Therapy and Surgery

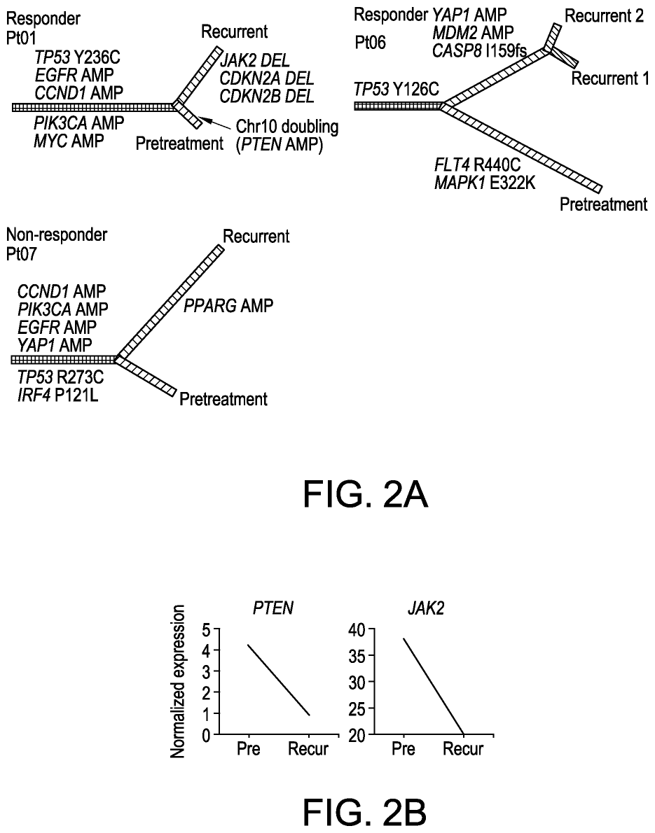

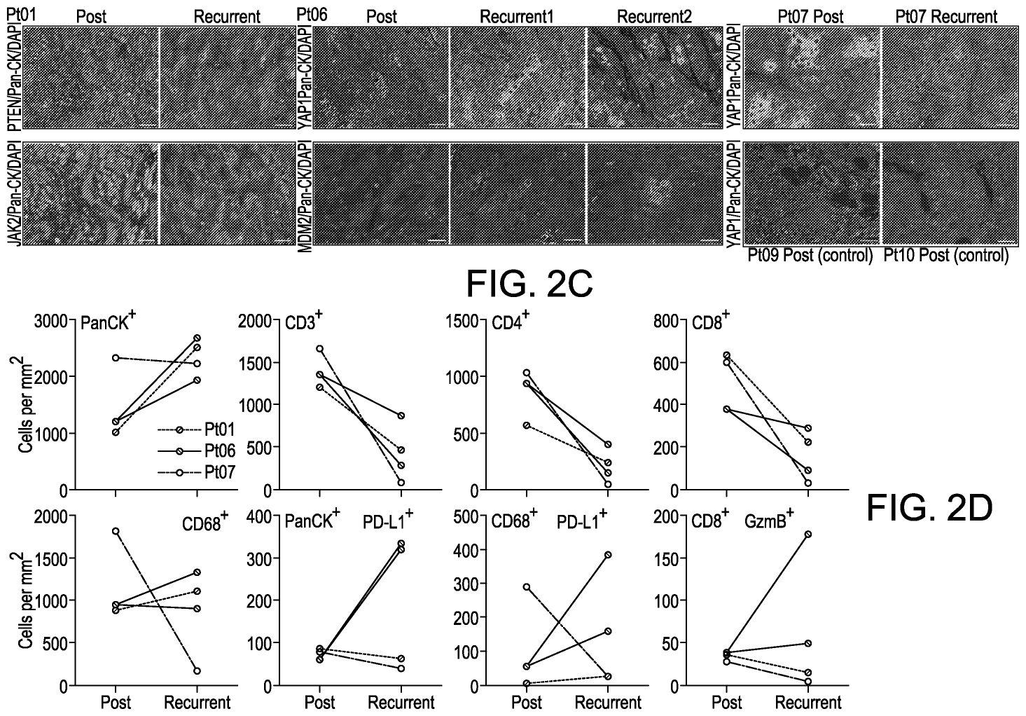

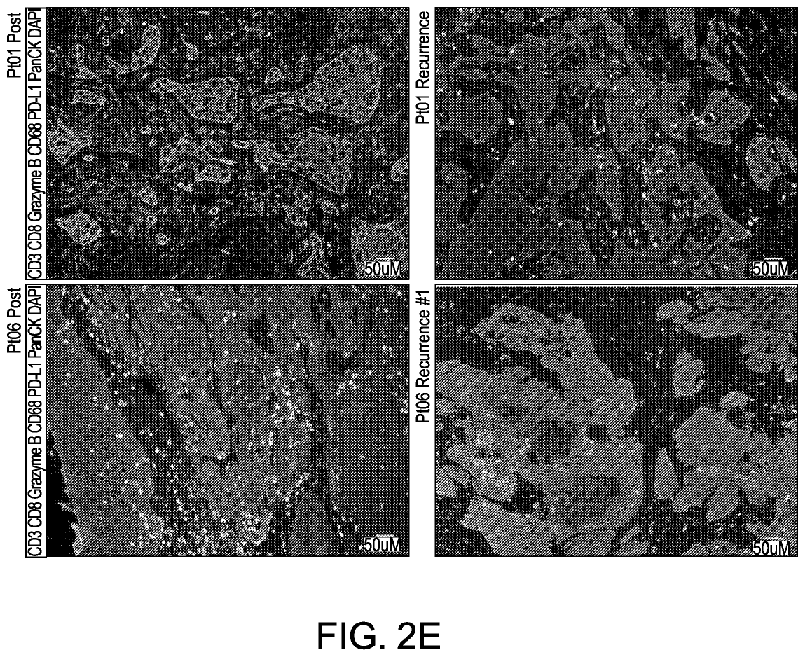

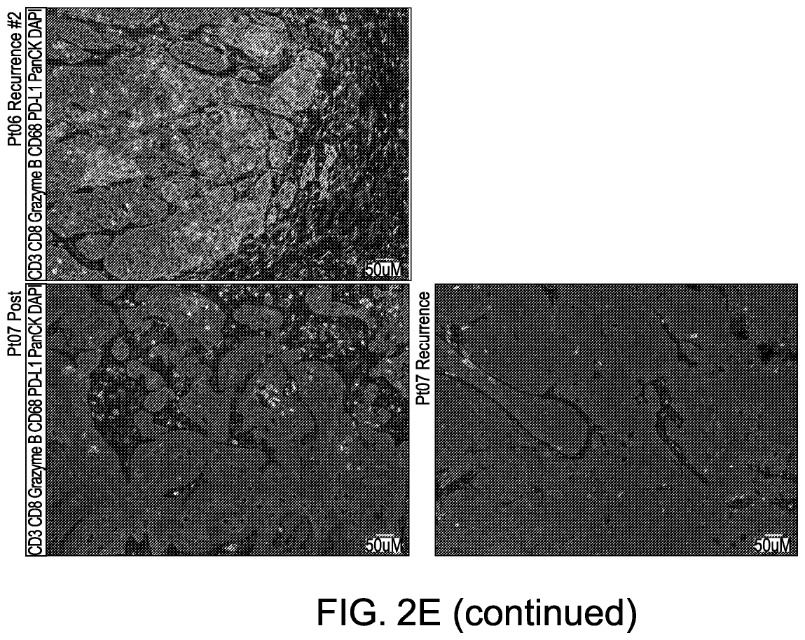

We further exploited WES data to retrace the evolutionary trajectories of OCSCCs from normal epithelial cells to malignant tumors before treatment with neoadjuvant nivolumab and then to recurrent tumors post-neoadjuvant therapy and post-surgery. The phylogenetic trees for two responders and one non-responder were constructed (FIG. 2A). In patient 1, neoadjuvant PD-1 blockade (four doses) elicited a 45% reduction in tumor size (Table S2), despite a very low TMB pretreatment (FIG. 1A). However, the patient's tumor recurred (in the lung) 0.58 year after surgical excision. We compared WES data from this recurrent tumor to WES from the pretreatment tumor and patient-matched normal tissue and found that the pretreatment and recurrent tumors evolved in a branched manner. In addition, chromosome 10, where PTEN resides, was amplified, due to arm-sized duplication, before nivolumab treatment. However, in the recurrent tumor, PTEN copy number (CN) was neutral, indicating a loss relative to the pretreatment tumor. Moreover, in the recurrent tumor, we observed CN losses of CDKN2A, CDKN2B, and JAK2 (FIG. 2A). However, only PTEN and JAK2 displayed concordant DNA and RNA loss in the recurrent tumor (PTEN, ˜4 fold; JAK2, ˜2 fold) (FIG. 2B). By immunofluorescence (IF), we corroborated PTEN and JAK2 protein-level reduction specifically in tumor cells of the recurrent tumor (FIG. 2C). Given emerging reports of mechanistic links between PTEN loss and innate anti-PD-1 resistance¹⁷⁻¹⁹, we speculate that PTEN loss promotes tumor recurrence in patients after post-neoadjuvant anti-PD-1 therapy and surgery. Also, we posit that the CN gain pretreatment may contribute to innate responsiveness of this tumor, despite its low TMB.

In another responding patient (Pt6), neoadjuvant PD-1 blockade elicited a 30% reduction in tumor size (Table S2). After the residual tumor was excised, the patient relapsed in 1.91 years with two recurrent tumors. As in the case of patient 1, evolution of pretreatment and recurrent tumors followed a branched pattern, where the ancestral clone harbored the same TP53 mutation (FIG. 2A). Notably, the two recurrent tumors originated from this ancestral clone with shared hits, namely YAP1 and MDM2 amplification. YAP1 post-transcriptional upregulation and nuclear translocation in tumor cells have been implicated in immune evasion during MAPK-targeted and anti-PD-1 therapies.²⁰⁻²² Also, MDM2 amplification, which has been linked to hyper-progression on anti-PD-1 therapy²³, can be targeted by small molecule inhibitors to improve anti-PD-1 responsiveness and T cell killing of cancer cells.^{24,25} Corresponding to these gDNA amplification events, both YAP1 and MDM2 protein levels were elevated in the tumor cells of recurrent (vs. post-treatment) tumors, with the YAP1 protein up-regulation being both cytoplasmic and nuclear in recurrent tumor #1 and largely nuclear in recurrent tumor #2 (FIG. 2C). Also relevant to recurrence may be loss of the FLT4 mutation in both recurrent tumors (FIG. 1D), suggesting that the missense FLT4 mutations that are enriched in responders may be gain-of-function mutations.

In a non-responding patient (Pt7), tumor recurred 0.73 years after neoadjuvant nivolumab and surgery (Table S2). Patient 7 was deceased within 1.5 months after clinical relapse. Despite a 26% increase in tumor size after neoadjuvant nivolumab therapy, the tumor that recurred after definitive surgery followed a branched evolutionary pattern, suggesting that some level of immune-editing occurred despite the lack of radiographic and pathologic response. As shown in FIG. 2A, YAP1 amplification predated the most recent ancestral tumor clone, suggesting a role in innate resistance. Consistent with preexisting YAP1 amplification, the YAP1 protein level was elevated in the tumor cells of both post-treatment and recurrent tumors in Pt7, in contrast to the YAP1 levels in the post-treatment tumors of Pt9 and Pt10 (which served as controls) (FIG. 2C). Interestingly, PPARG amplification was private to the recurrent tumor (FIG. 2A). Given that amplification-driven overexpression of PPARG, in concert with RXRa activation, has been shown to confer partial resistance to immunotherapy by impairing CD8 T cell infiltration in muscle-invasive bladder cancer²⁶, PPARG amplification may complement YAP1 amplification to tip the balance toward further immune evasion.

Using multiplex immunofluorescence (mIF), we also characterized the concurrent evolution of intratumoral immune microenvironment at the histologic level (quantifications in FIG. 2D and representative images in FIG. 2E). The panel consisted of antibodies against pan-cytokeratin (panCK), CD3, CD8, CD68, PD-L1, and granzyme B (GzmB). Based on whole-tissue quantification, we observed, expectedly, that panCK⁺ tumor cells (per mm2 of tissue) increased in the recurrent tumor of Pt1 and both recurrent tumors of Pt6 (vs. matched, post-treatment surgical tumors), since both Pt1 and Pt6 were responders (FIG. 2D). In contrast, there was little change in the density of panCK⁺ tumor cells in the recurrent tumor of Pt7, a non-responder (FIG. 2D). In patient-matched comparisons, all recurrent tumors displayed a significant decrease in total CD3⁺ T cells, which corresponded to a decrease in both CD4⁺ and CD8⁺ T cells (FIGS. 2D and 2E). This observation further supports the aforementioned notion that some level of immune-editing occurred despite the lack of radiographic and pathologic response in Pt7.

We observed additional recurrence-specific features compared with tumors on anti-PD-1 neoadjuvant therapy (FIGS. 2D and 2E). In Pt1, recurrence was associated with a loss of CD8⁺ T cell cytolytic activity, as defined by a reduced GzmB level, and increase in CD68⁺ macrophages. There was minimal change in PD-L1 expression in both tumor cells and macrophages. In Pt6, recurrence was associated with a gain in the level CD8⁺ GzmB⁺ T cells. However, there was a concurrent induction in the levels of both PD-L1+ tumor cells and macrophages. Thus, the combination of reduced overall T cell infiltration and increased PD-L1 immune checkpoint expression may have resulted in relapses. In Pt7, recurrence was deplete of immune cells, suggestive of an immune desert.

### Pre- and Post-Treatment Transcriptional Signatures of Response Patterns

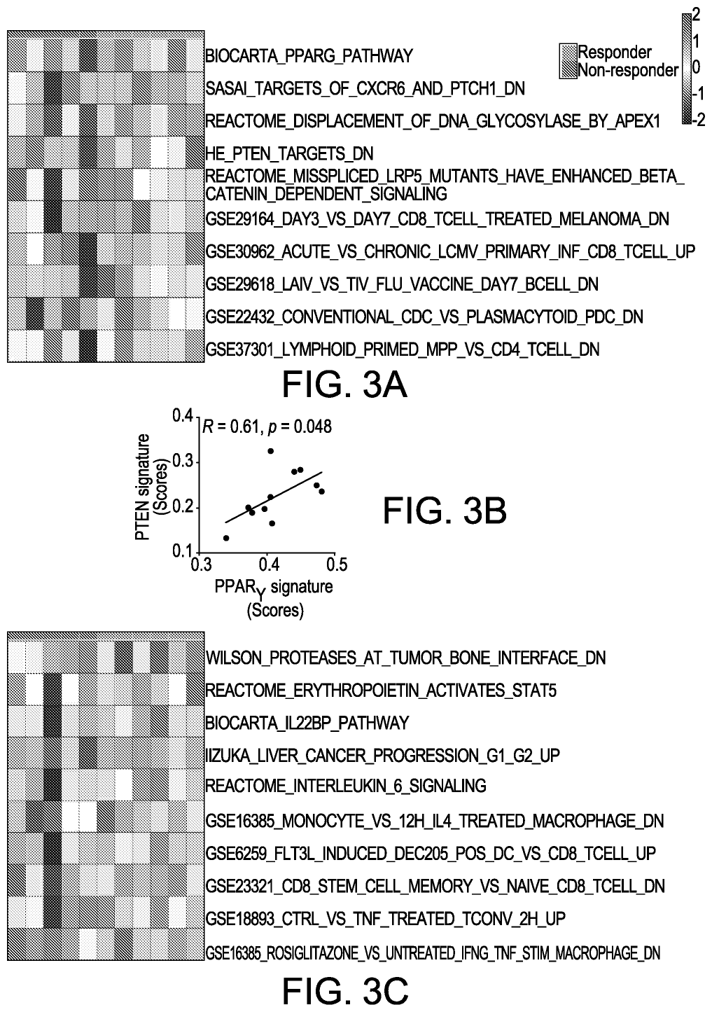

We analyzed RNA-seq data generated from 11 pairs of matched, pre- and post-tumors for statistically significant differential enrichment of 10,401 gene sets (MSigDB) between the responding and non-responding tumors, either before or after neoadjuvant nivolumab therapy. Among the two groups of pretreatment tumors, two potentially functionally relevant processes were differentially enriched (FIG. 3A). First, genes down-regulated in the intestine after the tissue-specific knockout of PTEN (HE_PTEN_TARGETS_DN) were negatively enriched among the non-responding pretreatment tumors, suggesting lower PTEN gene dosage or activity with innate anti-PD-1 resistance. Second, responsive pretreatment tumors were positively enriched for PPARg pathway genes. PPARg signaling increases PTEN activity.^{27,28} Consistent with this functional link, we detected a highly positive correlation of enrichment scores between these two gene sets (FIG. 3B). Furthermore, among the post-treatment tumors, two gene sets related to de-differentiation and stemness of cancer cells, namely IIZUKA_LIVER_CANCER_PROGRESSION_G1_G2_UP²⁹ and REACTOME_INTERLEUKIN_6_SIGNALING³⁰, were enriched in responders (FIG. 3C).

We next investigated gene signatures previously reported to be associated with ICB response in our cohort. By comparing the transcriptomic profiles among the pretreatment tumors, we observed that the enrichment of published signatures (effector T cell signature³¹, a six-gene/FNg signature (IFNg-6)³², and a cytolytic activity signature³³) and the PD-L1 RNA levels generally decreased from pretreatment tumors that displayed partial responses, to those that displayed stable disease, and then to those that displayed progressive disease, although the differences were not statistically significant (Figure S3A). Enrichment levels of the effector T cell signatures in the pretreatment tumor were negatively correlated with pathology-based changes in tumor sizes after treatment (Figure S3B). Based on RNA-seq data, we then estimated the subtypes of infiltrating immune cells by CIBERSORTX and observed that pretreatment CD8⁺ T cell infiltration levels were negatively correlated with pathology-based changes in tumor sizes after treatment (R<−0.5, Figure S3C). Thus, pretreatment tumors with higher intra-tumoral CD8⁺ T cell levels or pronounced enrichment of effector T cell signatures trended in patients with greater responses to neoadjuvant PD-1 blockade. Additionally, levels of pretreatment enrichment of a PTEN signature and intra-tumor CD8+ T cells estimated by CIBERSORTX did not associate with improved RFS and OS (Figure S3D and S3E). Moreover, there was no evidence that the enrichment levels of a PTEN signature among the pretreatment tumors were related to CD8⁺ T cell infiltration levels (Pearson correlation, R=0.06, p>0.87) or enhancement of the effector T cell signature (Pearson correlation, R=0.09, p>0.80).

### Relationships Between TCRβ Clonotypes and Tumor Response

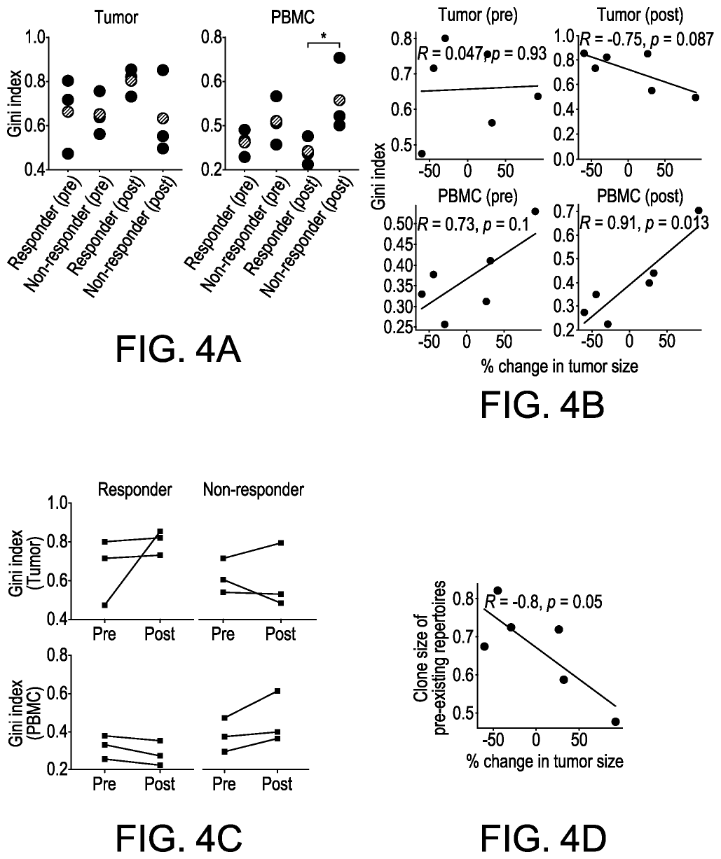

To understand how TCRβ clonotypes (pre- and post-nivolumab treatment, within peripheral blood and tumor) track with response patterns, we selected available patient-matched PBMCs and tumors from three responders vs. three non-responders (Tables S2 and S3) to generate gDNA-based TCRβ sequencing. As expected, greater overlaps of productive CDR3 amino acid sequences among patient-matched samples was observed (Figure S4). Clonality, characterized by the Gini index, was not different in tumors of responders vs. non-responders, before or after neoadjuvant nivolumab therapy (FIG. 4A). However, within PBMCs, T cell clonality was significantly elevated in the non-responders after neoadjuvant nivolumab therapy (FIG. 4A). Consistently, T cell clonality in the peripheral blood post-treatment was positively correlated (p=0.013) with changes in tumor size (FIG. 4B). From an analysis of patient-matched TCRβ clonotypes pre-vs. post-nivolumab treatment, we noted opposite patterns in the tumor vs. PBMC in responders vs. non-responders (FIG. 4C). Responding tumors either maintained (2 of 3) or harbored (1 of 3) increased TCRβ clonality after treatment, whereas 2 of 3 non-responding tumors lost TCRβ clonality after treatment (FIG. 4C). In contrast, PBMCs of responders (3 of 3) lost T cell clonality, whereas PBMCs of non-responders (3 of 3) gained T cell clonality (FIG. 4C). The prior data (FIGS. 4A to 4C) supportive of intratumoral T cell clonal expansion after tumor shrinkage led us to investigate the clonal origins of expanded clones. 52.83% of the preexisting TCRβ clones (repertoires shared by pre- and post-tumors) and 22.18% of novel clonotypes (post-tumor specific clones) were detectable in the pretreatment PBMCs across patients, but these detection rates of intratumoral T cell clonotypes within pretreatment peripheral blood were not significantly different in responders vs. non-responders. Importantly, the clone sizes of preexisting clones in post-treatment tumors were significantly and negatively correlated with changes in tumor size, suggesting that preexisting intratumoral T cell clones that expanded in response to neoadjuvant nivolumab treatment led to tumor shrinkage (FIG. 4D).

### Pretreatment Peripheral Blood T_{REG} and TH17 Imbalance Signals Tumor Progression

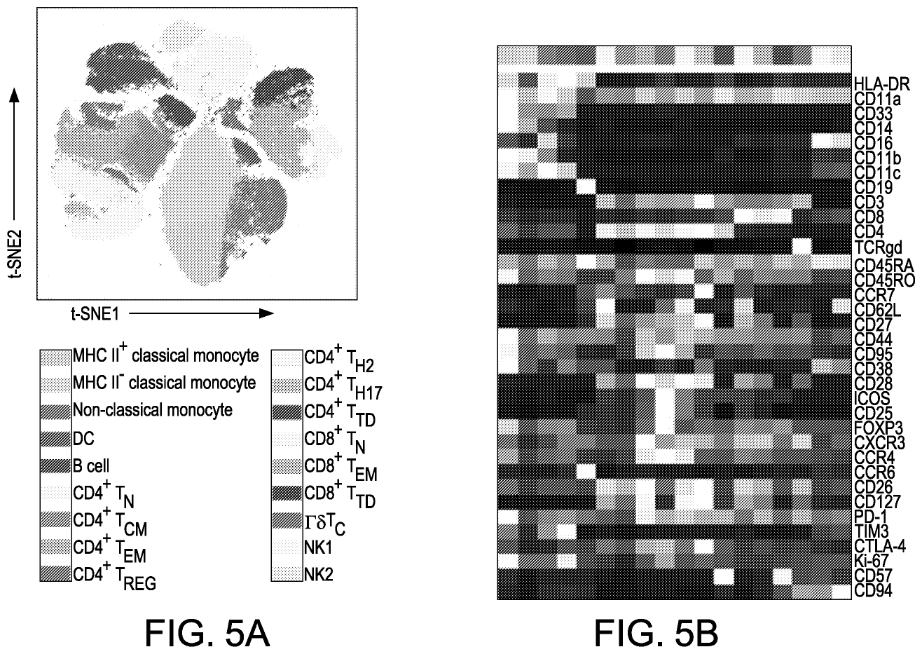

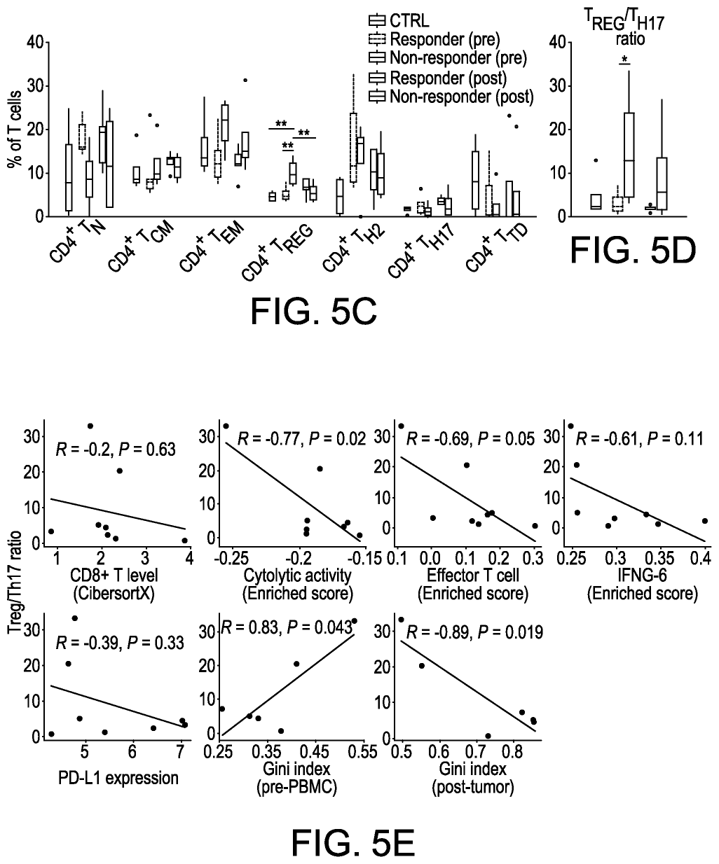

To evaluate differences in immune cell populations in the peripheral blood between responders (n=5) vs. non-responders (n=4), we used CyTOF to analyze PBMCs collected pre- and post-treatment (at the time of surgery) and PBMCs from healthy donors (n=4). By clustering analysis, we identified 18 immune cell populations (FIGS. 5A and 5B), including three CD8⁺ T cell (naïve or T_{N}. T effector memory or T_{EM}, and T terminally differentiated or T_{TD}); seven CD4⁺ T cell (naïve or T_{N}, T central memory or T_{CM}, T_{EM}, regulatory T or T_{REG}, T helper 2 or T_{H2}. T helper 17 or T_{H17}, and T_{TD}); gamma delta T cell or γδ T cell; three monocyte (MHC II⁺ classical, MHC II-classical, and non-classical monocytes); two NK (NK-1, CD62L⁻ and NK-2, CD62L⁺); B cell; and dendritic cell (DC) subpopulations. T cells (CD4⁺ and CD8⁺ subsets) were most abundant in healthy donors', responders' and non-responders' PBMCs (Figure S5A). Moreover, after neoadjuvant nivolumab treatment, the DC subpopulation was greatly compromised in the non-responder (vs. responder) group. Pretreatment, the level of B cells was significantly higher (vs. that in healthy donors) in the non-responder group (Figure S5A).

We then evaluated differences in the most abundant T cell subpopulations (FIGS. 5C and S5B). Importantly, we observed a significantly higher level of CD4⁺ T_{REG} cells in the pretreatment blood of non-responders (vs. those in normal donors, pretreatment responders, or post-treatment non-responders) (FIG. 5C). In this context, the level of FOXP3⁺ T_{REG} cells was observed to be significantly higher in the pretreatment peripheral blood of non-responders (vs. responders) to PD-1 blockade in patients with non-small cell lung carcinoma.³⁴ Interestingly, after neoadjuvant nivolumab treatment, T_{REG} levels increased in the responders but decreased in the non-responders. Contrary to T_{REG}, CD4⁺ T_{H17} trended higher in pretreatment PBMCs of responders (vs. non-responders). Considering their opposing functional impacts on antitumor CD8⁺ T cell immunity, we calculated the T_{REG}/T_{H17} ratio in each group and treatment time point. Notably, a six-fold higher T_{REG}/T_{H17} ratio was observed in the pretreatment PBMCs of the non-responder (vs. responder) group (FIG. 5D). Also, pretreatment peripheral blood T_{REG}/T_{H17} ratios were negatively correlated with both cytolytic activity and effector T cell signatures in the pretreatment tumors; positively correlated with TCRβ clonality in the pretreatment blood; but negatively correlated with TCRβ clonality in the post-treatment tumors (FIG. 5E). Furthermore, patients with lower pretreatment blood T_{REG}/T_{H17} ratios tended to display improved RFS and OS, although the differences did not reach statistical significance (Figure S5C). Hence, elevation of the pretreatment peripheral blood T_{REG}/T_{H17} ratio may be predictive of lack of response and reduced survival benefit after neoadjuvant anti-PD-1 therapy.

### Discussion

Earlier studies to understand innate response or resistance to anti-PD-1 therapy in advanced human malignancies pointed to the importance of tumor mutational burden^{35,36}, T cell infiltration into tumor cores³⁷, intratumoral immune suppressive processes as well as cellular differentiation states³⁵. In patients with HPV-negative, locally advanced, treatment-naïve OCSCCs treated with neoadjuvant nivolumab, we identified TMB, mutations in specific genes (CDKN2A, FLT4, YAP1), intratumoral PPARg/PTEN signaling, and peripheral blood T_{REG}/T_{H17} ratio as putative pretreatment predictive biomarkers.

TMB was not different between responders and non-responders. However, higher TMB was predictive of improved RFS. Two patients (Pt1 and Pt12) with low-TMB pretreatment tumors displayed tumor responses. Pt12 was lost to follow-up after 0.28 years. However, patient 1 relapsed within 0.58 years after treatment but remains alive as of most recent follow-up after 3.2 years, suggesting that other factors such as a high pretreatment PTEN gene dosage could have compensated for the low TMB. Consistent with this hypothesis, a recurrent tumor from patient 1 displayed loss of PTEN CN, transcript, and protein levels. Moreover, CDKN2A loss-of-function mutations were observed at a higher-than-expected frequency among non-responders. In advanced melanoma, innate resistance to anti-PD-1 therapy trended with CCND1 CN gain and CDKN2A CN loss.³⁸ Within subsets (acral melanoma and melanoma of unknown primary), this association was significant. In clinical melanoma, progressive tumors on ICB lose senescence-inducing genes such as CDKN2A.³⁹ Mechanistically in preclinical models, Cdkn2a deletion abrogated ICB-elicited tumor control, suggesting induction of tumor cell senescence may be important to prevent tumor progression of clones that escape immune-mediated tumor cell cytotoxicity.³⁹ It is currently unclear whether FLT4 mutations enriched in the pretreatment tumors of responder patients are gain- or loss-of-function mutations. The lack of detection of the FLT4 mutant allele in both recurrent tumors, from a responder patient whose pretreatment tumor harbored a FLT4 mutation, supports FLT4 missense mutations as gain-of-function mutations. FLT4 (VEGFR3) promotes lymphangiogenesis, although little is known regarding its mutational impacts on cancer hallmarks. Recent studies of clinical colorectal carcinoma and clinical melanoma have correlated lymphatic vessel density and lymphatic gene expression to cytotoxic T cell density and immune infiltration, respectively.^{40,41} In mice lacking dermal lymphatics, fewer immune cells infiltrate melanoma.⁴² Thus, tumor-elicited lymphangiogenesis may promote immune infiltration, perhaps by increasing trafficking of tumor antigens and antigen-presenting cells to draining lymph nodes and facilitating T cell priming. Furthermore, the finding of co-enriched PPARg/PTEN gene sets in the responding, pretreatment tumors implicates COX-2 as a co-target, as PPARg serves to adaptively temper COX-2-mediated inflammation.⁴³ The action of PPARg may be mediated, at least in part, by PTEN upregulation^{27,28}, which is supported here by the positive correlation between as PPARg and PTEN signature enrichments among pretreatment OCSCC tumors.

Despite our small sample size, retracing the evolutionary histories of several patients' disease provided further clues to gene alterations potentially driving the patterns of initial responses and subsequent post-surgical recurrences. Several observations link determinants of initial response patterns with those of recurrence. As examples, CDKN2A deletions were enriched among initial non-responders and detected in a recurrent tumor. A PTEN signature was enriched among responders, and its deletion detected in a recurrent tumor. YAP1 amplification occurred in the pretreatment tumor of a non-responder; its amplification characterized two recurrent tumors in an initially responding patient. FLT4 missense mutations were enriched among initial responders. Among one of these responders whose pretreatment tumor carried a FLT4 mutation, both matched recurrent tumors had lost the FLT4 mutant allele. PPARG and PTEN signature co-enrichment characterized initial responses. However, PPARG amplification was detected in a case of recurrence (from a non-responder), suggesting a context-dependent role.

By analyzing the TCRβ repertoires, we observed a trend toward higher diversity (lower clonality) in the pretreatment PBMCs of responders vs. non-responders, and this difference reached statistical significance after treatment. Similarly, in post-treatment PBMCs, higher diversity (lower clonality) was associated with tumor shrinkage. In patient-matched analysis of PBMCs, TCR diversity increased after treatment in responders but decreased in non-responders. In the context of these findings, high pretreatment diversity of TCR clones in the peripheral blood has been associated with improved outcomes in patients with melanoma treated with anti-PD-1 or -CTLA-4 therapy.^{44,45} In a recent study of patients with non-small cell lung carcinoma treated with anti-PD-L1 therapy, induction of TCR diversity on day 15 was significantly associated with improved OS.⁴⁶ Thus, systemic TCR repertoire diversification pre- and especially post-neoadjuvant nivolumab treatment in OCSCCs is associated with tumor response and may be predictive of improved survival, which warrants future testing. Moreover, expansion of the preexisting intratumoral TCR repertoire after neoadjuvant nivolumab treatment was positively and significantly correlated with tumor shrinkage. We also identified the level of FOXP3⁺ T_{REG} cells to be significantly higher in the pretreatment peripheral blood of non-responders (vs. responders). After neoadjuvant nivolumab treatment, T_{REG} levels increased in the responders but decreased in the non-responders. Since PD-1 signaling restrains the suppressive activity of T_{REG}⁴⁷, this pattern suggests that T_{REG} targeting may improve responsiveness to neoadjuvant anti-PD-1 therapy. In addition, determining the pretreatment ratio of T_{REG}/T_{H17} in peripheral blood may be an important component of pretreatment analytics to stratify patients for neoadjuvant anti-PD-1 therapy as well as for adjuvant treatment intensification vs. de-escalation.

Here, we used response vs. non-response status based on tumor size changes measured radiographically pre-nivolumab treatment and pathologically post-nivolumab treatment. Although there was a short time interval between the post-nivolumab radiographic and pathologic assessment (Figure S1), 0/12 patients had a partial response based on the former, but 4/12 had a partial response based on the latter (Table S2). This type of discrepancy has been noted before. For example, in patients with early-stage NSCLC, only two patients experienced a radiographic partial response, despite a high rate of major pathologic response, and two tumors which had increased in size post-treatment harbored minimal, residual tumor in the surgical specimen.⁴⁸ All tumors in this study that responded pathologically also displayed a reduction in tumor size radiographically. Thus, our pathology-enhanced response criteria facilitated this correlative analysis.

In three small cohorts, including this current cohort, neoadjuvant efficacies of PD-1 blockade in resectable OCSCC cover a range due to variations in treatment (one to four doses of either nivolumab or pembrolizumab) and evaluation protocols. One study (nivolumab, 2 doses)¹¹ reported 13% response based on RECIST and 54% pathologic responses, with one of 12 patients displaying a major pathologic response (>90%). Another study (pembrolizumab, 1 dose)¹² reported 44% pathologic response ≥10%, with no major pathologic response observed. In the clinical trial related to this study, we observed a 33% overall response rate based on pathology-enhanced RECIST. These early clinical findings warrant larger studies that should conform treatment and evaluation standards to facilitate validation of molecular biomarkers nominated here.

### REFERENCES

1. Topalian S L, et al. N Engl J Med 2012; 366 (26): 2443-54.
2. Liu J, et al. Cancer Discov 2016; 6 (12): 1382-99.
3. Blank C U, et al. Nat Med 2018; 24 (11): 1655-61.
4. Bray F, et al. CA Cancer J Clin 2018; 68 (6): 394-424.
5. Leemans C R, et al. Nat Rev Cancer 2018; 18 (5): 269-82.
6. Cooper J S, et al. N Engl J Med 2004; 350 (19): 1937-44.
7. Cancer Genome Atlas N. Comprehensive genomic characterization of head and neck squamous cell carcinomas. Nature 2015; 517 (7536): 576-82.
8. Burtness B, et al. Lancet 2019; 394 (10212): 1915-28.
9. Ferris R L, et al. N Engl J Med 2016; 375 (19): 1856-67.
10. Seiwert T Y, et al. Lancet Oncol 2016; 17 (7): 956-65.
11. Schoenfeld J D, et al. JAMA Oncol 2020; 6 (10): 1563-70.
12. Uppaluri R, et al. Clin Cancer Res 2020; 26 (19): 5140-52.
13. Knochelmann H M, et al. Cell Reports Medicine 2021; Accepted.
14. Chowell D, et al. Science 2018; 359 (6375): 582-7.
15. Stransky N, et al. Science 2011; 333 (6046): 1157-60.
16. Shukla S A, et al. Nat Biotechnol 2015; 33 (11): 1152-8.
17. George S, et al. Immunity 2017; 46 (2): 197-204.
18. Peng W, et al. Cancer Discov 2015; December 8: Epub ahead of print.
19. Zhao J, et al. Nat Med 2019; 25 (3): 462-9.
20. Hugo W, et al. Cell 2015; 162 (6): 1271-85.
21. Kim M H, et al. Cancer Immunol Res 2018; 6 (3): 255-66.
22. Yu M, et al., Mol Cell 2021; 81 (6): 1216-30 e9.
23. Kato S, et al. Clin Cancer Res 2017; 23 (15): 4242-50.
24. Fang D D, et al. J Immunother Cancer 2019; 7 (1): 327.
25. Sahin I, et al. Cell Death Discov 2020; 6:57.
26. Korpal M, et al. Nat Commun 2017; 8 (1): 103.
27. Bonofiglio D, et al. Clin Cancer Res 2005; 11 (17): 6139-47.
28. Patel L, et al. Curr Biol 2001; 11 (10): 764-8.
29. Iizuka N, et al. FEBS Lett 2005; 579 (5): 1089-100.
30. Iliopoulos D, et al. Proc Natl Acad Sci USA 2011; 108 (4): 1397-402.
31. Bolen C R, et al. Blood Adv 2017; 1 (22): 1884-90.
32. Ayers M, et al. J Clin Invest 2017; 127 (8): 2930-40.
33. Johnson B J, et al. Proc Natl Acad Sci USA 2003; 100 (5): 2657-62.
34. Kagamu H, et al. Cancer Immunol Res 2020; 8 (3): 334-44.
35. Hugo W, et al. Cell 2016; 165 (1): 35-44.
36. Rizvi N A, et al. Science 2015; 348 (6230): 124-8.
37. Tumeh P C, et al. Nature 2014; 515 (7528): 568-71.
38. Yu J, et al. Clin Cancer Res 2019; 25 (21): 6511-23.
39. Brenner E, et al. Nat Commun 2020; 11 (1): 1335.
40. Mlecnik B, et al. Sci Transl Med 2016; 8 (327): 327ra26.
41. TCGA. Genomic Classification of Cutaneous Melanoma. Cell 2015; 161 (7): 1681-96.
42. Lund A W, et al. J Clin Invest 2016; 126 (9): 3389-402.
43. Korbecki J, et al. Inflamm Res 2019; 68 (6): 443-58.
44. Hogan S A, et al. Cancer Immunol Res 2019; 7 (1): 77-85.
45. Postow M A, et al. J Immunother Cancer 2015; 3:23.
46. Naidus E, et al. Cancer Immunol Immunother 2021.
47. Tan C L, et al. J Exp Med 2021; 218 (1).
48. Forde P M, et al. N Engl J Med 2018; 379 (9): e14.
49. Hong A, et al. Cancer Discov 2021; 11 (3): 714-35.
50. Moriceau G, et al. Cancer Cell 2015; 27 (2): 240-56.
51. Shi H, et al. Cancer Discov 2014; 4 (1): 80-93.
52. Shi H, et al. Nat Commun 2012; 3:724.
53. Ramos A H, et al. Hum Mutat 2015; 36 (4): E2423-9.
54. Favero F, et al. Ann Oncol 2015; 26 (1): 64-70.
55. Kim D. et al. Nat Methods 2015: 12 (4): 357-60.
56. Anders S, et al. Bioinformatics 2015; 31 (2): 166-9.
57. Hanzelmann S, et al. BMC Bioinformatics 2013; 14:7.
58. Newman A M, et al. Nat Biotechnol 2019; 37 (7): 773-82.
59. Kotecha N, et al. Curr Protoc Cytom 2010; Chapter 10: Unit10 7.
60. Chen H, et al. PLOS Comput Biol 2016; 12 (9): e1005112.
61. Nazarov V I, et al. BMC Bioinformatics 2015; 16:175.

Throughout this application various publications are referenced. The disclosures of these publications in their entireties are hereby incorporated by reference into this application in order to describe more fully the state of the art to which this invention pertains.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A method of treating a subject in need of treatment for resectable head and neck cancer, the method comprising:
(a) assaying a biological sample obtained from the subject for at least one of:
(1) tumor mutational burden (TMB) in tumor cells,
(2) mutations in FLT4, PTEN, YAP1, and/or JAK2 in tumor cells,
(3) signature enrichment or mutational status in PTEN in tumor cells,
(4) ratio of regulatory T to TH17 cells in peripheral blood mononuclear cells (PBMCs),
(5) scoring of tumor-infiltrating cytolytic T cells;
(6) T cell receptor (TCR) diversity and clonality in tumors or in PBMCs at a first time point and at a second time point; and
(b) treating the subject with:
(1) neoadjuvant anti-PD-1/L1 therapy prior to surgical resection when the assaying of (a) detects:
(i) greater than 5.8 mutations per megabase (Mb) in tumor cells,
(ii) mutations in FLT4 in tumor cells,
(iii) signature enrichment of PTEN or wildtype PTEN,
(iv) a ratio of regulatory T cells to TH17 cells of ≤4 in PBMCs; and/or
(v) brisk tumor infiltration of cytolytic T cells; and
(2) surgical resection without neoadjuvant anti-PD-1 therapy when the assaying of (a) detects:
(i) less than 5.8 mutations per megabase (Mb) in tumor cells,
(ii) mutations in PTEN, YAP1, and/or JAK2 in tumor cells,
(iii) a ratio of regulatory T cells to TH17 cells of greater than 4 in PBMCs;
(iv) a reduction in TCR diversity or increase in TCR clonality in PBMCs at the second time point compared to the first time point; and/or
(v) non-brisk tumor infiltration of cytolytic T cells.

2. The method of claim 1, wherein the assaying of step (a) comprises detecting the ratio of regulatory T to TH17 cells in PBMCs, and is performed prior to anti-PD-1/L1 treatment and prior to surgical resection.

3. The method of claim 1, further comprising:
(c) repeating the assaying of (a) at or near the time of surgical resection and after neoadjuvant anti-PD-1/L1 therapy; and
(d) treating the subject with adjuvant anti-PD-1/L1 therapy following surgical resection when the repeated assaying of (c) detects:
(i) loss of FLT4 mutations,
(ii) loss of PTEN signature enrichment or wildtype status,
(iii) loss of brisk tumor infiltration of cytolytic T cells,
(iv) no significant change or decrease in the diversity of TCR repertoire in PBMCs, and/or
(v) no gain or expansion in the clonality of preexisting, intratumoral large-size T cell clonotypes.

4. The method of claim 1, further comprising co-treating the subject with a combination of anti-PD-1/L1 therapy and agents targeting or neutralizing regulatory T cells, stimulating TH17 cells, or reversing the functional impacts of PTEN and JAK2 loss-of-function mutations or YAP1 gain-of-function mutations or post-transcriptional alterations, when the assaying of (a) detects:
(i) less than 5.8 mutations per megabase (Mb) in tumor cells,
(ii) mutations in PTEN, YAP1, and/or JAK2 in tumor cells,
(iii) a ratio of regulatory T cells to TH17 cells of greater than 4 in PBMCs; and/or
(iv) non-brisk tumor infiltration of cytolytic T cells.

5. The method of claim 4, wherein the assaying of (a) is performed before neoadjuvant therapy or at the time of surgery.

6. The method of claim 4, wherein the agent that targets regulatory T cells is a CD25NIB antibody.

7. The method of claim 1, wherein the biological sample comprises tumor biopsies and PBMCs isolated from peripheral blood.

8. The method of claim 3, wherein the treating of step (d) further comprises radiochemotherapy.

9. The method of claim 1, wherein the head and neck cancer is HPV-negative squamous cell carcinoma.

10. The method of claim 1, wherein the head and neck cancer is oral-cavity squamous cell carcinoma.

11. The method of claim 1, wherein the assaying of step (a) is performed at a first time point and again at a second time point, and wherein the treating of step (b) comprises administering neoadjuvant anti-PD-1/L1 therapy prior to surgical resection when the assaying of (a) detects an increase in TCR diversity or reduction in TCR clonality in PBMCs at the second time point relative to the first time point, and/or an increase in total T cell clonotypes in the tumor at the second time point relative to the first time point.

12. The method of claim 11, wherein the increase in total T cell clonotypes in the tumor at the second time point relative to the first time point is an increase of greater than 5%.

13. The method of claim 5, wherein the agent that targets regulatory T cells is a CD25NIB antibody.
